(19) United States
(12) Patent Application Publication
(10) Pub. No.: US 2010/0203043 A1
(43) Pub. Date: Aug. 12, 2010

(54) TREATMENT AND DIAGNOSIS OF METASTATIC PROSTATE CANCER WITH INHIBITORS OF EPIDERMAL GROWTH FACTOR RECEPTOR (EGFR)

(76) Inventors: Anne H. Ree, Haslum (NO); Ase Bratland, Oslo (NO); Pieter Jacob Boender, Nijmegen (NL); Robby Ruijtenbeek, Utrecht (NL)

Correspondence Address: AMSTER, ROTHSTEIN & EBENSTEIN LLP, 90 PARK AVENUE, NEW YORK, NY, 10016, US

(21) Appl. No.: 12/450,743

(22) PCT Filed: Apr. 11, 2008
(86) PCT No.: PCT/EP2008/054432

§ 371 Date: Mar. 12, 2010

(30) Foreign Application Priority Data
Apr. 13, 2007 (DK) PA 2007 00547

(51) Int. Cl. A61K 39/395; A61K 31/5377; A61K 31/517; A61K 31/4706; A61K 38/09; A61P 35/00; A61P 35/04; G01N 33/53

(52) U.S. Cl. 424/133.1; 424/174.1; 514/234.5; 514/266.4; 514/313; 424/142.1; 514/15; 435/7.1; 435/7.92

(57) ABSTRACT

The present invention relates to a method for the treatment, prevention and/or diagnosis of metastatic prostate cancer. More specifically inhibitors of Epidermal Growth Factor Receptor (EGFR) are used in the preparation of a pharmaceutical composition for treating or preventing metastatic prostate cancer. The EGFR inhibitors can for instance be EGFR inhibitors, EGFR signaling inhibitors and/or inhibitors of kinases downstream of EGFR kinases. The EGFR inhibitors can also be used in detection, screening, prediction and treatment monitoring methods for metastatic prostate cancer.

### FIELD OF INVENTION

The present invention relates to a method for treating or preventing metastatic prostate cancer. More specifically the method comprises the use of a therapeutically effective amount of an EGFR inhibitor or EGFR signaling inhibitors or inhibitors of kinases downstream of EGFR kinases to a patient in need thereof.

### BACKGROUND

Metastatic prostate cancer is a leading cause of cancer morbidity and mortality. The skeleton is the principal organ for metastasis formation in prostate cancer, and bone metastases are often both painful and debilitating. Androgens are critical regulators of prostate carcinoma growth and progression, but most patients respond only temporarily to androgen ablation therapy, also at bone metastasis sites.

Skeletal metastases from prostate cancer are essentially osteoblastic, as apparent both radiographically and histopathologically, and as consistent with elevated serum level of bone-specific alkaline phosphatase, a marker of osteoblast proliferation, in patients with metastatic prostate cancer [Logothetis & Lin, 2005]. These observations implicate that the biological interaction between the prostate carcinoma cells and osteoblasts contributes to the metastatic progression of prostate cancer.

Insight into regulatory mechanisms underlying establishment of prostate carcinoma cells within an osteoblastic microenvironment might lead to more effective therapies for metastatic disease.

A cascade of events is required for prostate carcinoma cells to metastasize to bone. The questions of how features of prostate carcinoma cells influence osteoblasts and vice versa, and how these features facilitate formation of the typical skeletal lesions, are still elusive. Insight into the mechanisms underlying these processes will not only help to explain the predilection of prostate cancer—as opposed to other tumor entities—to an osteoblastic bone microenvironment, but could also lead to development of prediction biomarkers and/or preventive agents or more effective therapies for advanced prostate cancer.

Conceptually, the overall metastatic potential may depend on a set of cellular characteristics that also determine the carcinoma cells' affinity for the bone marrow microenvironment. The elucidation of regulatory mechanisms underlying colonization of bone, however, depends on a careful choice of experimental model and analytical technology.

From a clinical point of view, prostate cancer metastasis to bone is a lengthy and complex disease process, which makes it difficult to find adequate laboratory models to recreate all steps involved [Singh & Figg, 2005]. However, given that regulatory mechanisms implicated in the metastatic phenotype are evoked when the carcinoma cells settle within an osteoblastic microenvironment, an experimental setup addressing how osteoblastic cells may influence prostate carcinoma cell biology upon formation of bone metastasis was invented. As highlighted in a recent review [Singh & Figg, 2005], most of the experimental systems examining this phenomenon are based on rodent models. Importantly, the model systems used by us exclusively utilize cell types of human origin.

### SUMMARY OF THE INVENTION

To study the regulatory basis underlying establishment of prostate carcinoma cells within an osteoblastic microenvironment, an experimental model system and novel analytical technology were combined to obtain information about functional signaling pathways and networks involved.

We used the human, androgen-sensitive LNCaP prostate carcinoma cell line [Horoszewicz et al., 1983] in coculture with the human, osteoblast-derived OHS cell line [Fodstad et al., 1986], as previously described [Bratland et al., 2003], to simulate the direct cellular interaction.

Factors secreted by osteoblasts have been proposed to stimulate prostate carcinoma cells [Logothetis & Lin, 2005]. Monocultured LNCaP cells were therefore treated with OHS-conditioned medium to experimentally replicate the biological context of paracrine influence.

Androgens are critical regulators of prostate carcinoma progression; however, until recently, the regulatory program mediated by the androgen receptor in prostate cancer has been elusive [Dehm & Tindall, 2006]. To simulate the complex processes involved in aberrant activation of the androgen signaling axis in prostate cancer [Scher & Sawyers, 2005; Attard et al., 2006], our experimental setup included LNCaP cells treated with a synthetic androgen analog (R1881) to observe whether androgen receptor-mediated signaling pathways might differ from pathways activated by OHS-directed influence.

The network connectivity analysis revealed that only one signaling pathway, i.e., that mediated by EGFR, was activated by the influence of both osteoblastic cells and androgen treatment (Table 1, FIG. 1). Hence, these experimental data suggest that targeted inhibition of this particular signaling pathway will simultaneously ablate androgen-driven proliferation of prostate carcinoma cells as well as their survival responses to an osteoblastic microenvironment, thereby providing a biological rationale for first-line use of EGFR inhibition in systemic prevention or treatment of metastatic prostate cancer in the androgen-sensitive stage of the disease.

Moreover, the data suggest the use of EGFR expression, with or without concomitant expression of other tumor cell markers, for detection of circulating tumor cells in bone marrow from prostate cancer patients, also with localized and/or androgen-sensitive disease, as predictive marker for later development of skeletal metastatic disease.

We used two additional experimental setups as biological controls for the LNCaP/OHS cocultures; the first to provide cells that might be more representative of physiological osteoblasts and the second to model regulatory interactions between carcinoma and osteoblastic cells in the androgen-independent stage of prostate cancer.

Non-hematopoietic stem cells in bone marrow are capable of differentiating into a variety of tissue entities, including osteogenic cells of bone tissue [Giordano et al., 2007]. Incubation of mononuclear cells isolated from adult, human bone marrow with mesenchymal stem cell-stimulating medium followed by osteogenic differentiation medium [Colter et al., 2000; Peister et al., 2004] gave rise to cells with osteoblastic characteristics, for example mineral deposition and alkaline phosphatase-secreting activity. These in vitro-differentiated normal osteoblasts were cocultured with LNCaP cells.

Androgen-independent LNCaP-19 cells, which have been derived from LNCaP cells following continuous maintenance in steroid-depleted medium, have been shown to form epithelial-like cell clusters [Gustaysson et al., 2005]. These cells were cocultured with OHS cells.

EGFR-mediated signaling was found increased in LNCaP cells from coculture with osteoblastic cells that had been differentiated from normal, human mesenchymal stem cells, but not in LNCaP-19 cells that were cocultured with OHS cells.

Thus, a first aspect of the invention is a method of treating or preventing metastatic prostate cancer comprising administering a therapeutically effective amount of an EGFR inhibitor or EGFR signaling inhibitors or inhibitors of kinases downstream of EGFR kinases to a patient in need thereof.

A second aspect of the invention is use of an EGFR inhibitor or EGFR signaling inhibitors or inhibitors of kinases downstream of EGFR kinases for the preparation of a medicament for treating or preventing metastatic prostate cancer.

A third aspect of the invention is a pharmaceutical composition comprising an EGFR inhibitor or EGFR signaling inhibitors or inhibitors of kinases downstream of EGFR kinases for treatment of metastatic prostate cancer.

A fourth aspect of the invention, is a method of predicting an individual's response on EGFR inhibitor(s) or pharmaceutical composition(s), i.e. ex-vivo drug testing and response prediction based on validated biomarker(s) or biomarker profiles, comprising
a) Providing a sample from the individual;
b) determining, in the sample, a level of EGFR protein, a level of EGFR phosphorylation, a level of EGFR kinase activity, a level of an EGFR pathway related kinase protein, a level of an EGFR pathway related kinase activity, a profile of EGFR pathway related kinase activities and/or a phosphorylation level of an EGFR pathway related kinase; and
c) comparing the level(s) or profile determined in step b with (a) control value(s) or control profile from which the clinical outcome of the treatment is a priori known.

A fifth aspect of the invention is a method of detecting metastatic prostate cancer, predicting metastatic prostate cancer or monitoring treatment of metastatic prostate cancer in a an individual comprising
a) Providing a sample from the individual;
b) determining, in the sample, a level of EGFR protein, a level of EGFR phosphorylation, a level of EGFR kinase activity, a level of an EGFR pathway related kinase protein, a level of an EGFR pathway related kinase activity, and/or a phosphorylation level of an EGFR pathway related kinase; and
c) comparing the level(s) determined in step b with (a) control value(s) of control profiles.

As additional aspects of the invention are provided further diagnostic methods as well as kits for detecting metastatic prostate cancer, predicting metastatic prostate cancer or monitoring treatment of metastatic prostate cancer in a an individual.

### DETAILED DESCRIPTION

### Brief Description of the Drawings

FIG. 1. Interconnected signaling pathways activated in LNCaP cells by influence of osteoblastic cells or androgen treatment. Two pathway visualization systems were applied to the data set (Table 1), which resulted in almost identical network connectivity maps. The substrate annotations are derived from gene entries in SwissProt. The lines connecting nodes represent interactions of the following types: binding, expression, protein modification, and regulation. Red, yellow, and blue nodes: Substrates activated by the direct, paracrine, and androgenic LNCaP entities, respectively.

FIG. 2. ROC (Receiver Operating Characteristics) curve.

FIG. 3. Percentile plot.

### DISCLOSURE OF THE INVENTION

As described above, the present inventors have studied the regulatory basis underlying establishment of prostate carcinoma cells within an osteoblastic microenvironment using a model system allowing identification of activated intracellular signaling pathways.

The network connectivity analysis of phosphopeptide signatures revealed that only one signaling pathway, i.e., that mediated by EGFR, was activated by the influence of both osteoblastic cells and androgen treatment. These experimental data suggest that targeted inhibition of this particular signaling pathway will simultaneously ablate androgen-driven proliferation of prostate carcinoma cells as well as their survival responses to an osteoblastic microenvironment, thereby providing a biological rationale for first-line use of EGFR inhibition in systemic prevention or treatment of metastatic prostate cancer in the androgen-sensitive stage of the disease.

Moreover, the data also suggest the possible use of EGFR expression, with or without concomitant expression of other tumor cell markers, for detection of circulating tumor cells in bone marrow from prostate cancer patients, also with localized and/or androgen-sensitive disease, as predictive marker for later development of skeletal metastatic disease

Thus, in a first aspect, the invention provides a method of treating or preventing metastatic prostate cancer comprising administering a therapeutically effective amount of an EGFR inhibitor or EGFR signaling inhibitors or inhibitors of kinases downstream of EGFR kinases to a patient in need thereof.

Preferably, the treatment is initiated while the cancer is in the androgen-sensitive stage of the disease.

As prostate cancer very often forms metastases in bone, the method of the invention is particularly preferred for treating or preventing skeletal metastatic prostate cancer.

The treatment may be an adjuvant treatment following removal of the primary tumor, neoadjuvant prior to surgery or definitive radiotherapy, or concomitant with radiotherapy.

EGFR inhibitor or EGFR signaling inhibitors or inhibitors of kinases downstream of EGFR kinases

The EGFR inhibitor or EGFR signaling inhibitors or inhibitors of kinases downstream of EGFR kinases to be used with the method of the invention are preferably selected from the group consisting of small molecules, an antibody directed toward EGFR and an aptamer directed toward EGFR.

High-affinity aptamers can be generated using a process called SELEX. Antibodies can be generated by a variety of methods known to the man skilled in the art (display techniques, hybridoma technology etc.). The antibodies may be non-human in which case an immune response directed toward them is to be expected. The immune response is often non-desirable, but may in cases they may be desired, e.g. for rapid clearance.

Preferably the antibodies are monoclonal. Particular preferred antibodies are those that have already been approved as therapeutics. Preferably, the antibody is selected from group consisting of cetuximab (Erbitux®), panitumumab (Vectibix™) and EMD7200.

In one embodiment, all antibodies with known anti-EGFR activity are included.

In a preferred embodiment, small molecules that target EGFR are selected from the group consisting of gefitinib N-(3-chloro-4-fluoro-phenyl)-7-methoxy-6-(3-morpholin-4-ylpropoxy)quinazolin-4-amine (Iressa®), erlotinib N-(3-ethynylphenyl)-6,7-bis(2-methoxyethoxy)quinazolin-4-amine (Tarceva®), and EKB-569.

In one embodiment, all small molecules with known anti-EGFR activity are included.

Dosis and regimen for preferred EGFR inhibitors or EGFR signaling inhibitors or inhibitors of kinases downstream of EGFR kinases are:

### Antibodies:

Cetuximab (Erbitux®) inj. 1. dose 400 mg/m2, followed by 250 mg/m2 weekly
Panitumumab (Vectibix™) inj. 6 mg/kg every 14 days
Matuzumab (EMD72000) inj. 800 mg weekly
Pertuzumab (Omnitarg™) inj. 1. dose 840 mg, followed by 420 mg every 21 days

### Small Molecular Tyrosine Kinase Inhibitors:

gefitinib (Iressa®) oral 250 (−500) mg*1
erlotinib (Tarceva®) oral 150 mg*1
EKB-569 oral 50 (−75) mg*1

In a preferred embodiment, the doses of the preferred EGFR inhibitors or EGFR signaling inhibitors or inhibitors of kinases downstream of EGFR kinases are reduced as compared to the above listed doses. Preferably, the dosis is reduced at least 10%, even more preferred at least 25% and most preferred at least 50%. A reduction of dosis may be particular feasible when EGFR inhibitors or EGFR signaling inhibitors or inhibitors of kinases downstream of EGFR kinases are combined with androgen ablation treatment.

Androgen ablation treatment.

In a preferred embodiment of the first aspect, the patient is further subject to androgen ablation treatment.

Androgen ablation treatment may be achieved surgical removal of the testicles of the patient.

In another embodiment, androgen ablation treatment comprises administrating an antiandrogen (testosterone antagonist or LHRH/GnRH analog).

Preferably, the antiandrogen is selected from the group consisting of flutamide (Eulexin), bicalutamide (Casodex) and nilutamide (Nilandron), leuprolin (enanton Depot®), buserelin (Suprefact depot) and triptorelin (Pamorelin®).

Dosis and regimen for preferred antiandrogens are:

### Testosteron Antagonists:

Bicalutamide (Casodex®) oral 50 mg*1
Flutamide (Eulexin®) oral 250 mg*3
Flutamide (Flutamid®) oral 250 mg*3

### LHRH/GnRH Analoges:

Goserelin (Zoladex®) inj 10.8 mg every 12th week
Leuprorelin (Enanton Depot®) inj 11.25 mg every 12th week
Leuprorelin (Procren Depot®) inj 11.25 mg every 12th week
Leuprorelin (Eligard®) inj 22.5 mg every 12th week
Buserelin (Suprefact Depot) inj 6.3 mg every 8th week
Triptorelin (Pamorelin®) inj 11.25 mg every 12th week

In a preferred embodiment, the doses of the preferred antiandrogens are reduced as compared to the above listed doses. Preferably, the dosis is reduced at least 10%, even more preferred at least 25% and most preferred at least 50%.

### Use of an EGFR Inhibitor or EGFR Signaling Inhibitor or Inhibitor of Kinases Downstream of EGFR Kinases for Preparing a Medicament

A second aspect of the invention is the use of an EGFR inhibitor or EGFR signaling inhibitors or inhibitors of kinases downstream of EGFR kinases for the preparation of a medicament for treating or preventing metastatic prostate cancer. The embodiments of the first aspect also apply to the second aspect of the invention.

A Pharmaceutical composition comprising an EGFR inhibitor or EGFR signaling inhibitors or inhibitors of kinases downstream of EGFR kinases

A third aspect of the invention is a pharmaceutical composition comprising an EGFR inhibitor or EGFR signaling inhibitor or inhibitor of kinases downstream of EGFR kinases for treatment of metastatic prostate cancer. The embodiments of the first aspect also apply to the third aspect of the invention, i.e. all the mentioned EGFR inhibitors or EGFR signaling inhibitors or inhibitors of kinases downstream of EGFR kinases apply and also treatment together with an antiandrogen.

Moreover, the pharmaceutical composition may comprise both the EGFR inhibitor or EGFR signaling inhibitor or inhibitor of kinases downstream of EGFR kinases and the antiandrogen.

Use of an EGFR Inhibitor or EGFR Signaling Inhibitor or Inhibitor of Kinases Downstream of EGFR Kinases for Preparing a Medicament

A second aspect of the invention is the use of an EGFR inhibitor or EGFR signaling inhibitors or inhibitors of kinases downstream of EGFR kinases for the preparation of a medicament for treating or preventing metastatic prostate cancer. The embodiments of the first aspect also apply to the second aspect of the invention.

### A Pharmaceutical Composition Comprising an EGFR Inhibitor or EGFR Signaling Inhibitors or Inhibitors of Kinases Downstream of EGFR Kinases

A third aspect of the invention is a pharmaceutical composition comprising an EGFR inhibitor or EGFR signaling inhibitor or inhibitor of kinases downstream of EGFR kinases for treatment of metastatic prostate cancer. The embodiments of the first aspect also apply to the third aspect of the invention, i.e. all the mentioned EGFR inhibitors or EGFR signaling inhibitors or inhibitors of kinases downstream of EGFR kinases apply and also treatment together with an antiandrogen.

Moreover, the pharmaceutical composition may comprise both the EGFR inhibitor or EGFR signaling inhibitor or inhibitor of kinases downstream of EGFR kinases and the antiandrogen.

### Detection, Prediction and Monitoring

A fourth aspect of the invention is a method of detecting metastatic prostate cancer, predicting metastatic prostate cancer or monitoring treatment of metastatic prostate cancer in a an individual comprising
a) Providing a sample from the individual;
b) determining, in the sample, a level of EGFR protein, a level of EGFR phosphorylation, a level of EGFR kinase activity, a level of an EGFR pathway related protein, a level of an EGFR pathway related protein kinase activity, and/or a phosphorylation level of an EGFR pathway related protein; and
c) comparing the level(s) determined in step b with (a) control value(s) of control profiles.

Optionally the activity profiles of the EGFR pathway related kinases are determined by testing multiple kinases in one assay or array.

A fifth aspect of the invention, is a method of predicting an individual's response on EGFR inhibitor(s) or pharmaceutical composition(s), i.e. ex-vivo drug testing and response prediction based on validated biomarker(s) or biomarker profiles, comprising
a) Providing a sample from the individual;
b) determining, in the sample, a level of EGFR protein, a level of EGFR phosphorylation, a level of EGFR kinase activity, a level of an EGFR pathway related protein, a level of an EGFR pathway related protein kinase activity, a profile of EGFR pathway related protein kinase activities and/or a phosphorylation level of an EGFR pathway related protein; and
c) comparing the level(s) or profile determined in step b with (a) control value(s) or control profile from which the clinical outcome of the treatment can is a priori known.

In a preferred embodiment the control value(s) is provided by determining the level of EGFR inhibitor or EGFR signaling inhibitor or inhibitor of kinases downstream of EGFR kinases in one or more healthy individuals. Details on a particular EGF receptor are available under GenBank Accession Number NM_{—}005228.

The said EGFR pathway related protein may be a kinase and/or a receptor. Particular proteins downstream of the EGFR, which are comprised by the definition of the term “EGFR pathway related protein”, may be selected from the group consisting of: ERBB2 (erythroblastic leukemia viral oncogene homolog 2, GenBank Acc. No. X03363, ERBB4 (erythroblastic leukemia viral oncogene homolog 4, GenBank Acc. No. L07868, MST1R (RON) macrophage stimulating 1 receptor (c-met-related tyrosine kinase), GenBank Acc. No. X70040, FAK (PTK2 protein tyrosine kinase 2) GenBank Acc. No. L13616, MET (HGFR) (hepatocyte growth factor receptor) GenBank Acc. No. M35073, RET (GDNF family receptor alpha 1 and 2 (GDNF)), GenBank Acc. No. AF038421, AF002700, NM_{—}001495, RAF1 (murine leukemia viral oncogene homolog 1), GenBank Acc. No. X03484, NM^{—}002880 and CREB1 (cAMP responsive element binding protein 1), GenBank Acc. No. M27691, JAK1 (Janus kinase 1) GenBank Acc. No. M64174, NM_{—}002227, IRS2 (insulin receptor substrate 2), GenBank Acc. No. AB000732, LCK (lymphocyte-specific protein tyrosine kinase), GenBank Acc. No. M36881, NM_{—}005356, PDPK1 (3-phosphoinositide dependent protein kinase-1), GenBank Acc. No. AF017995, EPHB1 (EPH receptor B1) GenBank Acc. No. L40636, NM_{—}004441, FAK2 (PTK2B protein tyrosine kinase 2 beta), GenBank Acc. No. U33284, NM_{—}004103, RASA (RAS p21 protein activator (GTPase activating protein) 1, 2, 3 and 4, GenBank Acc. No. NM_{—}002890, AF115573, NM_{—}006506, NM_{—}007368, AB011110, NM_{—}006989, ZAP70 (zeta-chain (TCR) associated protein kinase 70 kDa), GenBank Acc. No. L05148, CDK2 (cyclin-dependent kinase 2), GenBank Acc. No. M68520, LAT (linker for activation of T cells and linker for activation of T cells family, member 2), GenBank Acc. No. AF036905, AF257135, GSK3B (glycogen synthase kinase 3 beta), GenBank Acc. No. BC012760. The GenBank Accession numbers provide details on particular proteins downstream of the EGFR.

For the purpose of the present invention it may be preferred that the method comprises determining the expression profile of multiple EGFR pathway related protein, such as the expression profile of at least 3, at least 4, at least 5, at least 6, at least 7, at least 8, at least 9 or at least 10 EGFR pathway related proteins.

In further embodiments the read-out of control values comprises all or a subset of the set of peptide phosphorylations. Particular peptide phosphorylations are detailed in table 1.

A sixth aspect of the present invention relates to a method for determining whether an individual is likely to have metastatic prostate cancer, the method comprising determining a first parameter representing the level of EGFR protein and/or the level of EGFR kinase activity and/or EGFR pathway related kinase activity profiles as determined by testing multiple markers in samples. The method further comprises indicating the individual as having a high likelihood of having metastatic prostate cancer if the parameter is at or beyond a discriminating value and indicating the individual as unlikely of having metastatic prostate cancer if the parameter is not at or beyond the discriminating value.

The discriminating value is a value which has been determined by measuring the parameter in both a healthy control population and a population with known metastatic prostate cancer thereby determining the discriminating value which identifies the metastatic prostate cancer population with either a predetermined specificity or a predetermined sensitivity based on an analysis of the relation between the parameter values and the known clinical data of the healthy control population and the cancer patient population, [such as it is apparent from the detailed discussion in the examples herein]. The discriminating value determined in this manner is valid for the same experimental setup in future individual tests.

It is not relevant to give an exact threshold value. A relevant threshold value can be derived from the ROC (Receiver Operating Characteristics) curves which are drawn up in the application. These curves give the correlation between sensitivity and specificity and the sensitivity/specificity for any threshold value can be derived from the ROC curve.

A threshold value resulting in a high sensitivity results in a lower specificity and vice versa. If one wants to detect all prostate cancers with certainty, then the specificity will be lower and some false positives will be included. If one wants to be sure to only detect truly prostate cancers, then a number of prostate cancer would never be identified.

It is thus up to the individual diagnostic department to determine which level of sensitivity/specificity is desirable and how much loss in specificity is tolerable. The chosen threshold level could be dependent on other diagnostic parameters used in combination with the present method by the individual diagnostic department.

The specificity or sensitivity is to be chosen by the entity performing the diagnosis from a professional judgement of the degree of specificity/sensitivity is desirable, according to the discussion above and the threshold level of EGFR being determined from the ROC curve. The specificity/sensitivity is thus pre-determined and cannot be given as a fixed number because the specificity/sensitivity may vary depending on overall scope of the diagnostic procedure.

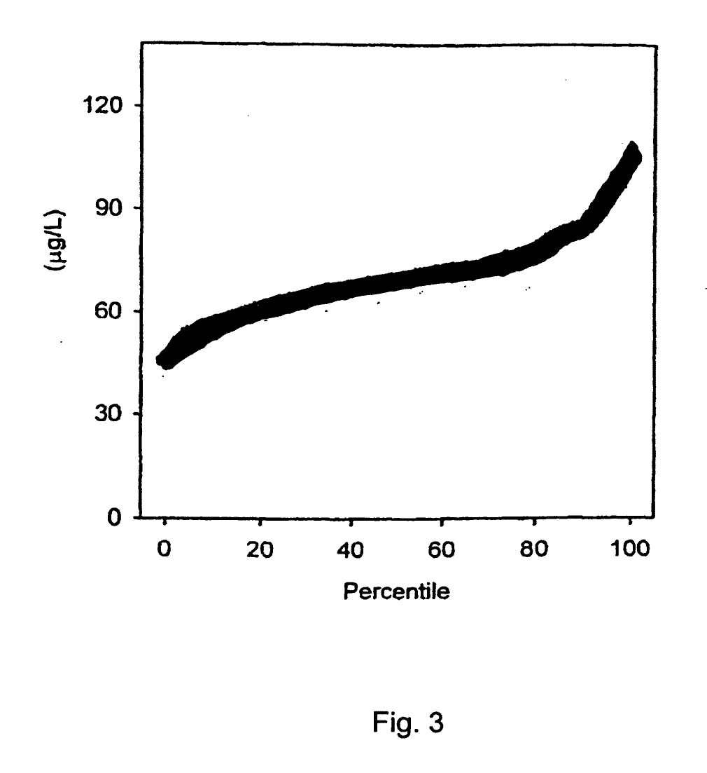

An example of a percentile plot of the EGFR levels in plasma from all metastatic prostate cancer patients and from healthy blood donors as presented in FIG. 3.

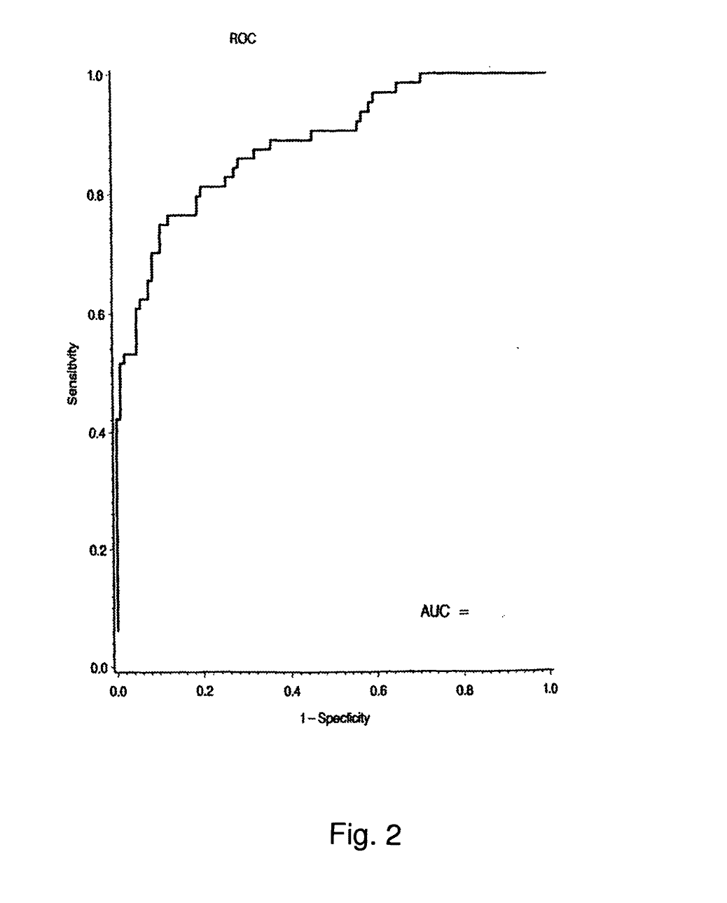

If a higher or lower sensitivity or specificity is desired, the cut-off value can be changed. This is illustrated in FIG. 2 showing a ROC example curves of EGFR in a sample from metastatic prostate cancer patients. Any other information which can be derived from these ROC curves falls within the scope of the present invention.

Accordingly, a method is provided for screening an individual for metastatic prostate cancer, the method comprising:
a) determining, in the sample of said individual, a level of EGFR protein, a level of EGFR phosphorylation, a level of EGFR kinase activity, a level of an EGFR pathway related protein, a level of an EGFR pathway related protein kinase activity, and/or a phosphorylation level of an EGFR pathway related protein; and
b) constructing a percentile plot of EGFR concentrations, levels of EGFR phosphorylation, levels of EGFR kinase activity, concentrations of an

EGFR pathway related protein, levels of phosphorylation of an EGFR pathway related protein and/or levels of an EGFR pathway related protein kinase activity, in a non-metastatic prostate cancer population;
c) constructing a ROC (receiver operating characteristics) curve based on EGFR concentrations, levels of EGFR phosphorylation, levels of EGFR kinase activity, concentrations of an EGFR pathway related, levels of phosphorylation of an EGFR pathway related protein and/or levels of an EGFR pathway related protein kinase activity determined in a non-metastatic prostate cancer population and on EGFR concentrations, levels of EGFR phosphorylation, levels of EGFR kinase activity, concentrations of an EGFR pathway related protein, levels of phosphorylation of an EGFR pathway related protein and/or levels of an EGFR pathway related protein kinase activity determined in a metastatic prostate cancer population;
d) selecting a desired sensitivity;
e) determining from the ROC curve the specificity corresponding to the desired sensitivity;
f) determining from the percentile plot the EGFR concentration value, the EGFR phosphorylation level value, the EGFR kinase activity value, the concentration value of an EGFR pathway related protein, the phosphorylation level value of an EGFR pathway related protein and/or the EGFR pathway related protein kinase activity levels value corresponding to the determined specificity; and
g) indicating the individual as likely to have metastatic prostate cancer if the concentration of EGFR, the levels of EGFR phosphorylation, the levels of EGFR kinase activity, the concentrations of an EGFR pathway related protein, the levels of phosphorylation of an EGFR pathway related protein and/or the levels of an EGFR pathway related protein kinase activity in the sample of the individual is equal to or higher than said EGFR concentration value, the EGFR phosphorylation level value, the EGFR kinase activity value, the concentration value of an EGFR pathway related protein, the phosphorylation level value of an EGFR pathway related protein and/or the EGFR pathway related protein kinase activity levels value corresponding to the determined specificity, and indicating the individual as unlikely to have metastatic prostate cancer if the concentration of EGFR, the levels of EGFR phosphorylation, the levels of EGFR kinase activity, the concentrations of an EGFR pathway related protein, the levels of phosphorylation of an EGFR pathway related protein and/or the levels of an EGFR pathway related protein kinase activity in the sample of the individual is lower than the EGFR concentration value, the EGFR phosphorylation level value, the EGFR kinase activity value, the concentration value of an EGFR pathway related protein, the phosphorylation level value of an EGFR pathway related protein and/or the EGFR pathway related protein kinase activity levels value corresponding to the determined specificity.

A related aspect of the invention provides a method for screening an individual for metastatic prostate cancer, the method comprising:
a) determining, in the sample of said individual, a level of EGFR protein, a level of EGFR phosphorylation, a level of EGFR kinase activity, a level of an EGFR pathway related protein, a level of an EGFR pathway related protein kinase activity, and/or a phosphorylation level of an EGFR pathway related protein; and
b) constructing a percentile plot of EGFR concentrations, levels of EGFR phosphorylation, levels of EGFR kinase activity, concentrations of an EGFR pathway related protein, levels of phosphorylation of an EGFR pathway related protein and/or levels of an EGFR pathway related protein kinase activity determined in a non-metastatic prostate cancer population and on EGFR concentrations, levels of EGFR phosphorylation, levels of EGFR kinase activity, concentrations of an EGFR pathway related protein, levels of phosphorylation of an EGFR pathway related protein and/or levels of an EGFR pathway related protein kinase activity determined in a metastatic prostate cancer population;
c) selecting a desired specificity;
d) determining from the percentile plot the EGFR concentration value, the EGFR phosphorylation level value, the EGFR kinase activity value, the concentration value of an EGFR pathway related protein, the phosphorylation level value of an EGFR pathway related protein and/or the EGFR pathway related protein kinase activity levels value corresponding to the desired specificity; and
e) indicating the individual as likely to have metastatic prostate cancer if the concentration of EGFR, the levels of EGFR phosphorylation, the levels of EGFR kinase activity, the concentrations of an EGFR pathway related protein, the levels of phosphorylation of an EGFR pathway related protein and/or the levels of an EGFR pathway related protein kinase activity in the sample of the individual is equal to or higher than said EGFR concentration value the EGFR phosphorylation level value, the EGFR kinase activity value, the concentration value of an EGFR pathway related protein, the phosphorylation level value of an EGFR pathway related protein and/or the EGFR pathway related protein kinase activity levels value corresponding to the determined specificity, and indicating the individual as unlikely to have metastatic prostate cancer if the concentration of EGFR, the levels of EGFR phosphorylation, the levels of EGFR kinase activity, the concentrations of an EGFR pathway related protein, the levels of phosphorylation of an EGFR pathway related protein and/or the levels of an EGFR pathway related protein kinase activity in the sample of the individual is lower than the EGFR concentration value, the EGFR phosphorylation level value, the EGFR kinase activity value, the concentration value of an EGFR pathway related protein, the phosphorylation level value of an EGFR pathway related protein and/or the EGFR pathway related protein kinase activity levels value corresponding to the determined specificity.

A further aspect of the invention provides method for screening an individual for metastatic prostate cancer, the method comprising determining in a sample from said individual a level of EGFR protein, a level of EGFR phosphorylation, a level of EGFR kinase activity, a level of an EGFR pathway related protein, a level of an EGFR pathway related protein kinase activity, and/or a phosphorylation level of an EGFR pathway related protein, and indicating the individual as likely to have metastatic prostate cancer if said level is equal to or higher than the respective level measured in a non-metastatic prostate cancer population, and indicating the individual as unlikely to have metastatic prostate cancer if said level is lower than the respective level in a non-metastatic prostate cancer population.

In a particular embodiment the method comprises determining:
i) the level of EGFR protein, the level of EGFR phosphorylation, and/or the level of EGFR kinase activity; and
ii) the level of at least one (such as at least 2, at least 3, at least 4, at least 5, at least 6, at least 7, at least 8, at least 9 or at least 10) EGFR pathway related protein(s), the level of at least one (such as at least 2, at least 3, at least 4, at least 5, at least 6, at least 7, at least 8, at least 9 or at least 10) EGFR pathway related protein kinase activity(ies), and/or the phosphorylation level of at least one (such as at least 2, at least 3, at least 4, at least 5, at least 6, at least 7, at least 8, at least 9 or at least 10) EGFR pathway related protein(s).

The method can be applied to an unselected population, but more appropriately to a population already identified as having an increased risk of developing prostate cancer, e.g. individuals with a genetic disposition, individuals who have been exposed to carcinogenic substances, individuals with increased serum levels of prostate-specific antigen (PSA), individuals with cancer-predisposing non-malignant diseases, individuals with one or more family members with prostate cancer, or individuals with a prior resection of an early prostate cancer.

In a preferred embodiment relating to screening/diagnostic methods of the invention the individual is a member of a population not already identified as having an increased risk of developing (metastatic) prostate cancer.

According to another preferred embodiment the individual is a member of a population not already identified as having an increased risk of developing (metastatic) prostate cancer.

In yet another preferred embodiment the individual is a member of a population already identified as having an increased risk of developing (metastatic) prostate cancer.

In particular, the individual may have a genetic disposition for (metastatic) prostate cancer, may have been exposed to carcinogenic substances or may have a (metastatic) prostate cancer-predisposing non-malignant disease.

Further, the individual may be selected from the group consisting of an individual who had any types of precursors to (metastatic) prostate cancer, and an individual with one or more family members with (metastatic) prostate cancer.

It will be understood that the metastatic prostate cancer may be selected from the group consisting of any relevant localised disease, either androgen sensitive or androgen resistant.

In another preferred embodiment, the sample is provided from bone marrow of the patient. The sample may also be blood or tissue, such as a tissue biopsy.

In a currently preferred embodiment testing of EGFR protein in the sample and/or determining EGFR kinase activity in the sample and/or determining EGFR related kinase activity profiles by testing multiple markers assaying is by microarray analysis. In currently preferred embodiments the microarray analysis is a three-dimensional flow-through solid support comprising through-going channels. Preferably, the solid support is a metal oxide support.

As the skilled person will appreciate, the determination of the concentration of EGFR in a sample of the individual is performed by means of an immuno assay or an activity assay. In particular, the immuno assay may be an ELISA, a western-blot or cytochemistry, and the activity assay may be based on substrate phosphorylation.

In a preferred embodiment, the sample is derived from red bone marrow of the patient or from a biopsy or surgical material from the primary tumor. Preferably, the sample comprises tumor cells isolated from red bone marrow or from the primary tumor. Isolation may be done using immunomagnetic target cell segregation, as outlined in the examples section, or using other automated immunomagnetic isolation technologies.

The immunosegregated cells from the bone marrow sample comprise prostate carcinoma cells in the systemic circulation of the patient. These are usually adenocarcinoma cells, infrequently neuroendocrine carcinoma cells. Generally, procedures for the acquisition of such cells, such as cell flow cytometry will be known to the person of skills in the art. In particular, isolation of tumour cells by the use of immuno-magnetic beads is provided in the present application as a non-limiting illustrative example.

The positive detection of immunosegregated cell in the bone marrow sample indicates a circulating cell population with capacity to form bone metastasis, provided this cell population possesses expression or activity of certain biomarkers.

A further aspect of the present invention pertains to a kit for the detection of metastatic prostate cancer, for the prediction of metastatic prostate cancer or for the monitoring of metastatic prostate cancer in an individual. The kits according to the invention comprise reagents to be used for determining EGFR or EGFR pathway related protein expression, phosphorylation and/or kinase activity. I particular embodiments the kits of the invention comprise specific antibodies (monoclonal or polyclonal) raised against EGFR or EGFR pathway related kinases. In preferred embodiments, the antibodies are labeled with fluorescent or luminescent tags. The kits may comprise further reagents and/or solutions useful for the detection of EGFR or EGFR pathway related protein expression, phosphorylation and/or kinase activity in samples, such as detection of protein levels by immunocytochemistry or -histochemistry.

In currently preferred embodiments, the detection of metastatic prostate cancer, prediction of metastatic prostate cancer or the monitoring of metastatic prostate cancer involves kinase activity profiling. Kinase activity profiling can be performed using various technologies. Preferably, PamChip® technology (PamGene International B.V., www.pamgene.com) is used. The PamChip® peptide array technology is described in the examples of the present application and in WO 99/02266, WO 01&12846, WO 200402667, WO 03102585 which are all incorporated into the present application in their entirety.

It should be noted that embodiments and features described in the context of one of the aspects of the present invention also apply to the other aspects of the invention.

Throughout the present specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All patent and non-patent references cited in the present application, are hereby incorporated by reference in their entirety.

The invention will now be described in further details in the following non-limiting examples.

### EXAMPLES

### Example 1

### EGFR Signaling is Activated by the Influence of Both Osteoblastic Cells and Androgen Treatment

### Methods

Cell culture conditions. The LNCaP and OHS cell lines were routinely held in RPMI 1640 medium supplemented with 10% fetal bovine serum (FBS) and 2.0 mM glutamine, defined as growth medium. Different ratios of LNCaP cells to OHS cells had been tested in a series of cocultures to find the optimal culturing conditions [Bratland et al., 2003]. Seventy-two hours before start of experimental incubations, LNCaP and OHS cells were seeded in a 10:1 ratio in RPMI containing 2% charcoal-treated FBS and glutamine. After 48 h, this medium was changed to RPMI containing 0.5% charcoal-treated FBS and glutamine, defined as experimental medium, for another 24 h before experimental incubations were started (at time 0). Monocultures of LNCaP cells were identically incubated prior to experimental incubations.

Monocultures of LNCaP, as well as LNCaP/OHS cocultures, were seeded in a total number of 1.0×10⁶ cells in 75 cm2 cell flasks and invariably held in 10 ml medium throughout different incubations. At time 0 (start of the experimental incubations), monocultured LNCaP cells were refed with experimental medium supplemented with 100 nM R1881 (methyltrienolone; Life Science Products), a synthetic androgen analog, or with medium conditioned by OHS cells. The conditioned medium had been collected from OHS monocultures that had been seeded in a 10-fold higher number per volume medium compared to the corresponding cell number of the LNCaP/OHS cocultures, and grown for 48 h (relative to time 0) in experimental medium. This medium was subsequently diluted 1:10 in either fresh experimental medium or in medium obtained from standard LNCaP monocultures after 48 h of incubation (relative to time 0) in experimental medium, before the application onto monocultured LNCaP cells.

### Experimental Approach

Skeletal metastases from prostate cancer are essentially osteoblastic, as apparent both radiographically and histopathologically, and as consistent with elevated serum level of bone-specific alkaline phosphatase, a marker of osteoblast proliferation, in patients with metastatic prostate cancer [Logothetis & Lin, 2005]. These observations implicate that the biological interaction between the prostate carcinoma cells and osteoblasts contributes to the metastatic progression of prostate cancer. To study the regulatory basis for this heterotypic cellular interaction, a model system allowing identification of activated intracellular signaling pathways was established. We used the human, androgen-sensitive LNCaP prostate carcinoma cell line [Horoszewicz et al., 1983] in coculture with the human, osteoblast-derived OHS cell line [Fodstad et al., 1986], as previously described [Bratland et al., 2003], to experimentally address how osteoblastic cells may influence prostate carcinoma cell biology upon formation of bone metastasis.

To be able to analyze LNCaP signaling pathways activated by direct contact with OHS cells, LNCaP cells were isolated from cocultures by immunomagnetic selection [Forus et al., 1999, Fodstad et al., 2001, Bruland et al., 2005, Tveito et al., 2007]. This rapid and simple procedure enables specific selection of target cells for further analytical applications. Subsequent multiplex profiling of kinase activity was performed using flow-through, porous microarrays with peptide substrates (PamChip® peptide arrays; PamGene International B.V., www.pamgene.com), a novel platform that allows rapid, real-time measurements of phosphopeptide signatures generated by the biological samples.

Insight into regulatory mechanisms underlying establishment of prostate carcinoma cells within an osteoblastic microenvironment might eventually lead to more effective therapies for metastatic disease. Hence, from a therapeutic perspective, we compared the intracellular LNCaP signaling pathways activated by OHS influence with those induced after androgen treatment of LNCaP cells, to suggest whether androgen ablation therapy, as used empirically today, represents a biologically based strategy in prevention and systemic treatment of bone metastasis in prostate cancer.

### The Biological Model

From a clinical point of view, prostate cancer metastasis to bone is a lengthy and complex disease process, which makes it difficult to find adequate laboratory models to recreate all steps involved [Singh & Figg, 2005]. However, we have now developed an experimental model and analytical technology providing relevant information about functional signaling pathways and networks that might initiate this biological process, also within a therapeutic perspective.

In this study, we used human, androgen-sensitive prostate carcinoma cells (LNCaP), considered to represent a validated model for prostate cancer in androgen-sensitive stage, in coculture with human osteoblast-derived cells (OHS). The OHS cell line was originally established from a patient with aggressive osteosarcoma [Fodstad et al., 1986], and it might therefore be argued that it is not fully representative for physiological osteoblasts. However, formation of osteosclerotic (i.e., osteoblastic) lesions following intratibial OHS cell inoculation has previously been demonstrated by radiographic, scintigraphic, and morphologic assessments [Kjønniksen et al., 1994].

### Results

Culturing LNCaP cells with OHS cells, which were seeded in a 10:1 ratio to generate stable cocultures [Bratland 2003], caused substantial change in LNCaP morphology. The spindle-shaped feature of monocultured LNCaP cells was rapidly lost upon direct contact with the OHS cells. In coculture, both cell types appeared rounded, although cytoplasmic processes were still apparent on LNCaP cells. Cellular morphology of the OHS cells, however, seemed to be independent of the culturing conditions.

We have previously evaluated the immunomagnetic cell segregation method for selective isolation of target cells and demonstrated that target cell populations are highly enriched [Forus et al., 1999, Fodstad et al., 2001, Bruland et al., 2005, Tveito et al., 2007]. Although cell fractions binding the MOC-31 (anti-EPCAM) antibody were essentially absent of cells defined as contaminants (i.e., with <5 immunobeads bound to their surface), the analytical application (kinase activity profiling) might be sensitive to their possible presence.

Hence, we screened for phosphopeptide signatures generated by immunoselected as well as monocultured OHS cells. Notably, the resulting substrate phosphorylation patterns were rather similar for the two conditions, but clearly distinguishable from those generated by the LNCaP entities, which argues against significant contamination of OHS cells in MOC-31-positive cell preparations.

Based on numerous observations that prostate carcinoma cells are stimulated by osteoblast-derived factors [Logothetis & Lin, 2005], monocultured LNCaP cells were treated with OHS-conditioned medium to experimentally obtain the biological context of paracrine influence. The profiles of phosphorylated peptides acquired from LNCaP cells were essentially identical whether the OHS-conditioned medium was mixed with fresh medium or with medium conditioned by LNCaP monocultures, although the generated phosphorylation levels were slightly higher for all peptides, with one exception (phospho-RB1), on microarrays incubated with the cells treated with medium conditioned by both cell types. Interestingly, LNCaP morphology appeared unchanged under the influence of OHS-conditioned media.

Androgens are critical regulators of prostate carcinoma progression. Most patients respond to androgen ablation therapy, but only temporarily, also at bone metastasis sites. Until recently, the regulatory program mediated by the androgen receptor in prostate cancer has been elusive [Dehm & Tindall, 2006]. To simulate the complex processes involved in aberrant activation of the androgen signaling axis upon disease progression of prostate cancer [Feldman & Feldman, 2001, Attard et al., 2006], our experimental setting included LNCaP cells treated with a synthetic androgen analog (R1881, 100 nM) to observe whether androgen receptor-mediated signaling pathways might be different from those activated by OHS-directed influence.

The LNCaP samples were given denotations in accordance with their biological context: ‘direct’ (cells from LNCaP/OHS cocultures), ‘paracrine 1’ (cells treated with OHS-conditioned medium mixed with fresh medium), ‘paracrine 2’ (cells treated with OHS-conditioned medium mixed with medium conditioned by LNCaP cells), and ‘androgenic’ (cells treated with the synthetic androgen analog), in addition to baseline (untreated reference cells).

### Kinase Activity Profiling

To identify regulatory mechanisms underlying the affinity of prostate cancer to bone, the substrate phosphorylation state generated by each LNCaP entity (‘direct’, ‘paracrine’, ‘androgenic’) was calculated with respect to baseline. Based on the assumption that biologically relevant signaling events implicated in the metastatic phenotype require sustained activation, 48 h incubation times were used for all experimental LNCaP contexts. The identified substrates showed increases in phosphorylation level within a broad range (Table 1).

Table 1: List of peptides with increased phosphorylation levels generated by the various LNCaP entities. For each substrate, position of phosphorylation sites within the protein is indicated. ‘-’ denotes that the change in peptide phosphorylation level was not found to be significant. Fold changes relative to LNCaP baseline sample are listed

| | ‘direct’ | ‘paracrine’ | ‘androgenic’ |
|---|---|---|---|
| Phosphopeptide | fold change (log2) | | |
| EGFR_y1197 | 2.16 | 1.84 | 1.94 |
| EGFR_y1110 | 1.86 | 2.34 | — |
| MST1R_y1353 | 2.20 | 2.86 | — |
| MST1R_y1356/y1360 | 2.01 | 3.38 | — |
| FAK_y576/y577 | 1.03 | 1.39 | — |
| FAK2_y579/y580 | 0.85 | 1.21 | — |
| LAT_y200 | 2.94 | 3.49 | — |
| ZAP70_y492/y493 | 1.21 | 1.93 | — |
| RASA_y460 | 0.96 | 1.63 | — |
| RET_y1029 | — | 1.76 | — |
| IRS2_y919 | — | 5.56 | — |
| JAK1_y1022/y1023 | — | 2.05 | — |
| LCK_y394 | — | 1.91 | — |
| MET_y1230/y1234/y1235 | — | 1.57 | — |
| PDPK1_y9 | — | 1.82 | — |
| PDPK1_y373/y376 | — | 1.82 | — |
| EPHB1_y778 | — | 2.11 | — |
| ERBB2_y877 | — | 1.67 | — |
| ERBB2_y1248 | — | 1.56 | — |
| CDK2_t14/y_5 | — | 1.86 | — |
| RB1_s807/s811 | — | 3.39 | 3.07 |
| RAF1_s337/s338/y339/y340 | — | — | 1.14 |
| GSK3B_y216 | — | — | 1.15 |
| CREBl_y134/s133 | — | — | 1.47 |
| ERBB4_y1284 | — | — | 3.31 |
| CHRNB1_y390 | 0.95 | 1.57 | 1.57 |
| LTK_y772/y776/y777 | 0.96 | — | 1.20 |
| DDRl_y792/y796/y797 | 0.91 | — | — |
| MAPK10 | 0.98 | — | — |
| MAPK12_t183/y185 | 2.23 | — | — |
| PECAM1_y713 | 0.87 | 2.06 | — |
| PRRX2_y214 | 0.93 | 1.48 | — |
| ANXA1_y20/t23 | — | 2.41 | — |
| CD79A_y182/y188 | — | 1.75 | — |
| CTTN1_y477/y483 | — | 2.01 | — |
| CTTN1_y499 | — | 2.13 | — |
| ENO2_y43 | — | 1.85 | — |
| EPHA2_y772 | — | 1.99 | — |
| EPHA7_y608/y614 | — | 2.27 | — |
| EPOR_y368 | — | 2.08 | — |
| EPOR_y426 | — | 2.29 | — |
| FER_y714 | — | 1.66 | — |
| FES_y713 | — | 2.20 | — |
| FGFR2_y769 | — | 1.77 | — |
| FGFR3_y760 | — | 2.23 | — |
| FRK_y387 | — | 2.02 | — |
| LAT_y255 | — | 1.54 | — |
| NTRK2_y702/y706/y707 | — | 1.43 | — |
| PDGFRB_y579/y581 | — | 3.87 | — |
| PDGFRB_y716 | — | 1.78 | — |
| PIK3R1_y607/s608 | — | 2.09 | — |
| PXN_y31 | — | 1.88 | — |
| PXN_y118 | — | 2.07 | — |
| TEC_y519 | — | 1.68 | — |
| PFKFB1_s33 | — | — | 1.36 |
| PTPN11_y542 | — | — | 2.94 |
| SYN1_s9 | — | — | 1.20 |

Each individual phosphopeptide signature was considered to represent a subset of the information flow through the globally activated signaling network of the particular LNCaP entity. To dissect how this information was directed, and in accordance with current recommendations [Petricoin III et al., 2005], we applied bioinformatics analysis methodologies routinely used for analysis of gene expression microarrays. By using such an approach, we assumed that phosphorylation events that appeared simultaneously might be interlinked and provide information about pathway connectivity [Sevecka & MacBeath, 2006].

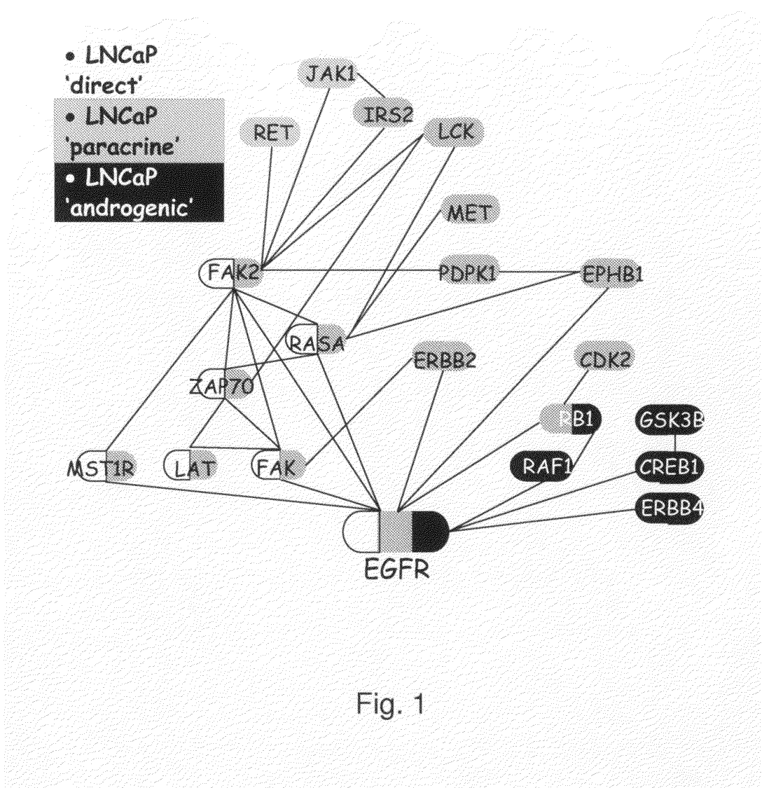

By applying these assumptions, the network interaction analysis omitted phosphorylated substrates that did not appear within any signaling pathway when defined by the interaction types delineated in Methods. Comparison of Table 1 with FIG. 1 indicates which phosphopeptides were left out. As illustrated by FIG. 1, the resulting network connectivity map showed that signaling pathways involved in cell adhesion and motility were activated in the ‘direct’ LNCaP entity, whereas the ‘paracrine’ entity additionally phosphorylated substrates involved in cell proliferation. Activation of similar but also completely unrelated proliferation pathways was observed with the ‘androgenic’ entity. Interestingly, only one signaling pathway, i.e., mediated by EGFR, was activated by the influence of both osteoblastic cells and androgen treatment.

### Conclusion

The network connectivity analysis revealed that only one signaling pathway, i.e., mediated by EGFR, was activated by the influence of both osteoblastic cells and androgen treatment. Based on these experimental data, we therefore hypothesize that targeted inhibition of this particular signaling pathway may simultaneously ablate androgen-driven proliferation of prostate carcinoma cells as well as their survival responses to an osteoblastic microenvironment, thereby providing a biological rationale for first-line use of EGFR inhibition in systemic prevention or treatment of metastatic prostate cancer in the androgen-sensitive stage of the disease.

Moreover, our data also suggest the possible use of EGFR expression, with or without concomitant expression of other tumor cell markers, for detection of circulating tumor cells in bone marrow from prostate cancer patients, also with localized and/or androgen-sensitive disease, as predictive marker for later development of skeletal metastatic disease.

### Example 2

### EGFR Signaling is Activated in Androgen-Sensitive Prostate Carcinoma Cells by the Influence of Normal, In Vitro-Differentiated Osteoblasts

### Introduction

Non-hematopoietic stem cells in bone marrow are capable of differentiating into a variety of tissue entities, including osteogenic cells of bone tissue [Giordano et al., 2007]. Incubation of mononuclear cells isolated from adult, human bone marrow with mesenchymal stem cell-stimulating medium followed by osteogenic differentiation medium [Colter et al., 2000; Peister et al., 2004] gave rise to cells with osteoblastic characteristics, for example mineral deposition and alkaline phosphatase-secreting activity. These in vitro-differentiated normal osteoblasts were cocultured with LNCaP cells.

### Results

We applied the immunomagnetic cell separation method for selective isolation of the LNCaP cells from the cocultured osteoblastic cells and subjected the isolated carcinoma cells to analysis by conventional western immunoblotting. Increased expression levels of EGFR phosphorylated on tyrosine 1173 were found in the isolated LNCaP cells.

### Conclusion

Activation of EGFR signaling was again found induced in prostate carcinoma cells under influence of osteoblastic cells, this time osteoblasts that had been differentiated from normal, human mesenchymal stem cells.

### Example 3

### Inability of Osteoblastic Cells to Activate EGFR Signaling in Androgen-Independent Prostate Carcinoma Cells

### Introduction

Androgen-independent LNCaP-19 cells had been derived from LNCaP cells following continuous maintenance in steroid-depleted medium [Gustaysson et al., 2005]. These cells were cocultured with OHS cells.

### Results

We applied the immunomagnetic cell separation method for selective isolation of the LNCaP-19 cells from the cocultured OHS cells and subjected the isolated carcinoma cells to analysis by conventional western immunoblotting. In clear contrast to the situation in androgen-sensitive prostate carcinoma cells (the maternal LNCaP cells), phosphorylation of EGFR on tyrosine 1173 was completely absent in LNCaP-19 cells that had been cocultured with osteoblastic cells.

### Conclusion

Given that EGFR is phosphorylated in the androgen-sensitive carcinoma cells upon influence of osteoblasts but not in the androgen-independent derivative cells, a functional androgen signaling axis [Scher & Sawyers, 2005; Attard et al., 2006] is probably permissive for activity of this particular pathway in prostate cancer.

Separately, EGFR may also facilitate androgen receptor-driven activity in prostate cancer at the level of target gene transcription [Gregory et al., 2004]. Androgen receptor pathway genes, identified by system-level analysis of gene expression in primary tumor specimens from therapy-naïve prostate cancer patients, were reported to be down-regulated in lymph node metastases from the patients [Hendriksen et al., 2006]. This finding further supports the assumption that the regulatory control by the androgen receptor on carcinoma cell biology is lost in the process of prostate cancer metastasis.

### Perspectives

Of importance, our experimental data suggests that targeted inhibition of signaling pathways directed by EGFR may simultaneously ablate androgen-driven proliferation of prostate carcinoma cells and the survival responses within an osteoblastic microenvironment. It equally provides a biological rationale for the use of EGFR inhibition in systemic prevention or treatment of metastatic prostate cancer in the androgen-sensitive stage of the disease. Intriguingly, the therapeutic concept of EGFR inhibition in hormone-refractory prostate cancer has recently been evaluated; however, in initial studies addressing the use of single-agent therapies in patients with androgen-resistant disease, neither a receptor-blocking antibody nor a small-molecular tyrosine kinase inhibitor showed clinically significant activity [de Bono et al., 2007; Agus et al., 2007; Canil et al., 2005]. Extrapolating from our data, we believe the loss of functional signaling governed by EGFR in androgen-independent prostate carcinoma cells may provide a biological explanation for the poor treatment efficacy in these trials. However, if exploitable in patients with therapy-naïve prostate cancer, inhibitory EGFR targeting might be incorporated into treatment schedules with a potential reduction of the alternative requirement of long-term androgen depletion, a reduction in relared side effects, and, intriguingly, the potential for an improvement in patient survival.

### Example 4

### Detection, Prediction and Monitoring

### Introduction

Clinical and experimental evidence suggests that epithelial tumor cells are able to disseminate to secondary organs at an early stage of primary tumor development. The red bone marrow represents an important indicator organ of hematogenous micrometastatic spread of carcinomas. According to current concept, however, disseminated tumor cells detected in the bone marrow are not able to grow as distant metastatic lesions unless they possess certain biological characteristics that may mediate proliferative responses upon colonization of the secondary organ.

The classical, experimental works on mechanisms of tumor metastasis demonstrated that only a small subset of cells within the parental population is capable of metastasizing and that the cellular composition of secondary tumors differs from that of the primaries. Cellular properties that are crucial for initiation of distant tumor growths, however, may be obscured by clonal heterogeneity of the fully established metastatic lesion. These findings are also in accordance with the contemporary concept of cancer stem cells. Hence, it is biologically relevant to analyze molecular properties that determine the affinity of epithelial tumor cells to the bone marrow and identify biomarkers that may correlate with the ability of distant growth and/or therapies directed against occult or established metastatic disease.

Immunomagnetic Target Cell Segregation from Bone Marrow

MOC-31 (IQ Corporation BV, Groningen, the Netherlands) is an IgG1 class antibody that binds to the EPCAM antigen, which is consistently expressed in most epithelial cells [de Jonge et al., 1993]. The antibody is conjugated to superparamagnetic monodisperse particles coated with polyclonal sheep-antimouse IgG particles (Dynabeads SAM-450; Dynal A.S., Oslo, Norway), as recommended by the manufacturer.

Samples of red bone marrow (˜15 ml) are acquired by aspiration from the upper iliac crest of prostate cancer patients. After Lymphoprep (Nycomed, Oslo, Norway) density gradient centrifugation (1000 g for 10 min), mononuclear cells from the interface layer are collected, washed, and resuspended in 1% human serum albumin in 0.9% NaCl (HSA/PBS), then counted and diluted to a final concentration of ˜10⁷ cells/ml for immunomagnetic separation.

All solutions and cell preparations are kept on ice during the whole procedure to avoid nonspecific binding of immunobeads. First, MOC-31-coated beads are added at a ratio of 10:1 to total number of suspended cells, and the suspensions are incubated for 30 min at 4° C. on a rotating mixer. The cells are subsequently diluted in HSA/PBS to a final volume of 3.0 ml and left in a magnet holder for 2 min, and the supernatants, containing unbound cells, are decanted. The remaining cell-bead rosettes, trapped on the wall of the test tubes by the magnet, are washed three times with surplus volume of HSA/PBS to remove any contaminating material. Of the remaining positive cell fractions of ˜200 μl, 20 μl aliquots are examined by light microscopy for the principal presence of cells with ≧5 immunobeads bound to their surface (i.e., cell-bead rosettes). Quality control of positive and negative cell fractions has been documented previously [Forus et al., 1999; Fodstad et al., 2001, Bruland et al., 2005, Tveito et al., 2007].

Characterization of Tumor Cells Isolated from Bone Marrow

Fluorescent latex microparticles (Molecular Probes Europe, Leiden, the Netherlands) are conjugated with different antibodies (against EGFR, ERBB2, ERBB4, MST1R (RON), FAK, MET (HGFR), RET, RAF and CREB1) and used in a double staining procedure to show that cells magnetically selected with SAM-450 beads coated with MOC-31 also bind latex particles with antibodies targeting one of the other epitopes/antigens, thus providing additional evidence that the rosetted cells are indeed tumor cells and that their presence in bone marrow might predict later development of bone metastatic disease.

### Immunocytochemistry for Detection of Circulating Tumor Cells in Bone Marrow

Immunocytochemistry is done on cytospins from mononuclear cells suspensions of bone marrow aspirates using the APAAP technique (DAKO, Copenhagen, Denmark). The detected cells are double-stained with different antibodies (against EGFR, ERBB2, ERBB4, MST1R (RON), FAK, MET (HGFR), RET, RAF and CREB1) to detect tumor cells that might predict later development of bone metastatic disease.

### Immunohistochemistry of Biopsy or Surgical Samples

Although a small subset only of cells within the primary tumor is capable of metastasizing, in accordance with the contemporary concept of cancer stem cells, it might be therapeutically relevant to identify cells within the primary tumor that are likely to metastasize. This might be accomplished by means of immunohistochemistry of biopsy or surgical specimens with different antibodies (against EGFR, ERBB2, ERBB4, MST1R (RON), FAK, MET (HGFR), RET, RAF and CREB1).

### Kinase Activity Profiling of Tumor Samples

To identify the regulatory basis for the metastasizing capacity of prostate carcinoma cells, this cell population may be analyzed for activated intracellular signaling pathways. This might be technically challenging, however, with a limited number of target cells available for the purpose. One possible analytical approach is described.

Tumor cells (isolated from biopsy or surgical material from the primary tumor or from bone marrow) are lysed in M-PER Mammalian Extraction Reagent containing Halt Phosphatase Inhibitor Cocktail and EDTA-free Halt Protease Inhibitor Cocktail (Pierce Biotechnology, Inc.). Reference lysates (baseline samples) are made from monocultured LNCaP cells or biopsy or surgical material of normal prostate tissue.

The peptide substrate array technology allows functional comparison of biological samples without prior knowledge of the activity pathways influenced by the experimental manipulations. The high-throughput format of the PamChip® technology (PamGene International B.V., www.pamgene.com) is based on the use of a porous, three-dimensional aluminum-oxide material as solid support for the substrates. The sample lysates are actively pumped through the interconnected capillary pores of the arrays to allow contact with the reactive surface, which is increased-500-fold compared to two-dimensional geometry arrays, for enzymatic reaction with the peptide substrates. The phosphorylation kinetics is therefore rapid and can be completed within few minutes, allowing the generation of spot images to be followed in real-time. Each array contains ˜140 peptides spotted in duplicate, and these peptides consist of 13, 14 or 15 amino acids with sites for phosphorylation, mainly tyrosine.

The image information is converted using BioNavigator software (PamGene International B.V.). For each spot on the array, signal intensity after background subtraction is calculated and used for further analysis. Data normalization of mean signal intensity from duplicate spots and subsequent comparison analyses are conducted using GeneSpring software (Agilent Technologies, www.home.agilent.com). All values are normalized to the calculated mean value of all substrate phosphorylation intensities in the baseline sample.

Several different pathway visualization systems can be used to create information about pathway connectivity (e.g., PathwayArchitect software (Stratagene Corp., www.stratagene.com; Strand Life Sciences Pvt. Ltd., www.avadis.strandgenomics.com), PathwayStudio software (Ariadne Genomics, www.ariadnegenomics.com). The peptide identifications can be visualized through a direct interaction network, defined to show all interactions between peptides that are of the following types: binding, expression, protein modification, and regulation. Pathways may also be created directly from the ResNet database (Ariadne Genomics) and ranked by the hypergeometric probability factor where all interactions are selected on the criterion of the highest number of proteins being involved in a linear pathway or sub-pathway.

### Systemic Therapy

Micrometastasis status has been proposed as an entry in the TNM classification system of the International Union Against Cancer (UICC) as a prognostic factor for several types of solid cancers. In patients with primary breast cancer the presence of bone marrow micrometastases is significantly associated with shorter survival, but is not an independent prognostic factor, as shown by short-term as well as long-term follow-up studies.

A recent publication on patients with localized prostate cancer treated with definitive radiotherapy (i.e., curatively intended therapy) showed that the presence of circulating tumor cells in bone marrow at time of diagnosis was associated with increased risk of developing distant metastases [Berg et al., 2007]. Hence, given that the right patient population is identified, adjuvant treatment after curatively intended therapy (surgery, radiotherapy, other therapeutic modalities) may be shown beneficial. This must be proven in prospective, randomized trails.

Moreover, since our experimental data show that the signalling pathway mediated by EGFR was activated by the influence of both osteoblastic cells and androgen treatment, we hypothesize that targeted inhibition of EGFR signalling may ablate prostate carcinoma cells' survival responses to an osteoblastic microenvironment as well as androgens. Hence, in a metastatic setting, palliative, systemic treatment with EGFR inhibitor or EGFR signaling inhibitor or inhibitor of kinases downstream of EGFR kinases, in the absence or presence of androgen ablation, might be beneficial, also in first-line setting (when the tumor is still androgen-sensitive).

### REFERENCES

Agus D B, Sweeney C J, Morris M J et al. Efficacy and safety of single-agent pertuzumab (rhuMAb 2C4), a human epidermal growth factor dimerization inhibitor, in castration-resistant prostate cancer after progression from taxane-based therapy. J Clin Oncol 2007; 25, 675-81.
Attard G, Sarker D, Reid A, Molife R, Parker C, de Bono JS. Improving the outcome of patients with castration-resistant prostate cancer through rational drug development. Br J Cancer 2006; 95, 767-74.
Berg A, Berner A, Lilleby W, Bruland O S, Fossa S D, Nesland J M, Kvalheim G. Impact of disseminated tumor cells in bone marrow at diagnosis in patients with nonmetastatic prostate cancer treated by definitive radiotherapy. Int J Cancer 2007; 120, 1603-9.
Bratland Å, Ragnhildstveit E, Bjørnland K, Andersen K, Maelandsmo G M, Fodstad Ø, Saatcioglu F, Ree A H. The metalloproteinase inhibitor TIMP-2 is down-regulated by androgens in LNCaP prostate carcinoma cells. Clin Exp Metastasis 2003; 20:541-7.
Bruland O S, Hoifodt H, Saeter G, Smeland S, Fodstad O. Hematogenous micrometastases in osteosarcoma patients. Clin Cancer Res 2005; 11:4666-73.
Canil C M, Moore M J, Winquist E et al. Randomized phase II study of two doses of gefitinib in hormone-refractory prostate cancer: a trial of the National Cancer Institute of Canada-Clinical Trials Group. 3 Clin Oncol 2007; 23, 455-60.
Colter D C, Class R, DiGirolamo C M, Prockop D J. Rapid expansion of recycling stem cells in cultures of plastic-adherent cells from human bone marrow. Proc Natl Acad Sci USA 2000; 97, 3213-8.
de Bono J S, Bellmunt J, Attard G et al. Open-label phase II study evaluating the efficacy and safety of two doses of pertuzumab in castrate chemotherapy-naïve patients with hormone-refractory prostate cancer. J Clin Oncol 2007; 25, 257-62.
Dehm S M, Tindall D J. Molecular regulation of androgen action in prostate cancer. Cell Biochem 2006; March 3 [Epub ahead of print].
de Jonge M W, Kosterink J G, Bin Y Y, Bulte J W, Kengen R A, Piers D A, The T H, de Leij L. Radioimmunodetection of human small cells lung carcinoma xenografts in the nude rat using 111in-labelled monoclonal antibody MOC-31. Eur 3 Cancer 1993; 29A:1885-90.
Feldman B J, Feldman D. The development of androgen-independent prostate cancer. Nat Rev Cancer 2001; 1, 34-45.
Fodstad Ø, Brøgger A, Bruland Ø, Solheim O P, Nesland J M, Pihl A. Characteristics of a cell line established from a patient with multiple osteosarcoma, appearing 13 years after treatment for bilateral retinoblastoma. Int J Cancer 1986; 38:33-40.
Fodstad Ø, Faye R, Høifødt HK, Skovlund E, Aamdal S. Immunobead-based detection and characterization of circulating tumor cells in melanoma patients. Recent Results Cancer Res 2001; 158:40-50.
Forus A, Høiføidt HK, Øverli G E T, Myklebost O, Fodstad Ø. Sensitive fluorescent in situ hybridisation method for the characterisation of breast cancer cells in bone marrow aspirates. J Clin Pathol Mol Pathol 1999; 52:68-74.
Giordano A, Galderisi U, Marino I R. From the laboratory bench to the patient's bedside: an update on clinical trials with mesenchymal stem cells. J Cell Physiol 2007; 211, 27-35.
Gregory C W, Fei X, Ponguta L A et al. Epidermal growth factor increases coactivation of the androgen receptor in recurrent prostate cancer. J Biol Chem 2004; 279, 7119-30.
Gustaysson H, Welén K, Damber J E. Transition of an androgen-dependent human prostate cancer cell line into an androgen-independent subline as associated with increased angiogenesis. Prostate 2005; 62, 364-73.
Gregory C W, Fei X, Ponguta L A et al. Epidermal growth factor increases coactivation of the androgen receptor in recurrent prostate cancer. 3 Biol Chem 2004; 279, 7119-30.
Hendriksen P J, Dits N F, Kokame K et al. Evolution of the androgen receptor pathway during progression of prostate cancer. Cancer Res 2006; 66, 5012-20.
Horoszewicz J S, Leong S S, Kawinski E, Karr J P, Rosenthal H, Chu T M, Mirand E A, Murphy G P. LNCaP model of human prostatic carcinoma. Cancer Res 1983; 43:1809-18.
Kjønniksen I, Winderen M, Bruland Ø, Fodstad Ø. Validity and usefulness of human tumor models established by intratibial cell inoculation in nude rats. Cancer Res 1994; 54, 1715-9.
Logothetis C J, Lin S H. Osteoblasts in prostate cancer metastasis to bone. Nat Rev Cancer 2005; 5:21-8.
Peister A, Melland J A, Wang M, Tucker H A, Prockop D J. Stable transfection of MSCs by electroporation. Gene Therapy 2004; 11, 224-8.
Petricoin III E F, Bichsel V E, Calvert V S, Espina V, Winters M, Young L, Belluco C, Trock B J, Lippman M, Fishman D A, Sgroi D C, Munson P J, Esserman Liotta L A. Mapping molecular networks using proteomics: a vision for patient-tailored combination therapy. 3 Clin Oncol 2005; 23, 3614-21.
Scher H I, Sawyers C L. Biology of progressive, castration-resistant prostate cancer: directed therapies targeting the androgen-receptor signaling axis. J Clin Oncol 2005; 32, 8253-61.
Sevecka M, MacBeath G. State-based discovery: a multidimensional screen for small-molecule modulators of EGF signaling. Nat Methods 2006; 3, 825-31.
Singh A S, Figg W D. In vivo models of prostate cancer metastasis to bone. J Urol 2005; 174, 820-6.
Tveito S, Maelandsmo G M, Hoifodt H K, Rasmussen H, Fodstad O, Specific isolation of disseminated cancer cells—a new method permitting sensitive detection of target molecules of diagnostic and therapeutic value. Clin Exp Metastasis 2007; in press.

1. A method of treating or preventing metastatic prostate cancer comprising administering a therapeutically effective amount of an EGFR inhibitor or EGFR signaling inhibitor or inhibitor of kinases downstream of EGFR kinases to a patient in need thereof.

2. The method of claim 1, wherein the treatment is initiated while the primary tumor is in the androgen-sensitive stage of the disease.

3. The method of claim 1, wherein the metastatic prostate cancer is in the androgen-sensitive stage of the disease.

4. The method of claim 1, wherein the metastatic prostate cancer is skeletal metastatic prostate cancer.

5. The method of claim 1, wherein the treatment is an adjuvant treatment following removal (surgery, radiotherapy, other therapy modalities) of the primary tumor.

6. The method of claim 1, wherein the EGFR inhibitor or EGFR signaling inhibitors or inhibitors of kinases downstream of EGFR kinases is selected from the group consisting of small molecules, an antibody directed toward EGFR and an aptamer directed toward EGFR.

7. The method of claim 6, wherein the small molecule is selected from the group consisting of gefitinib N-(3-chloro-4-fluoro-phenyl)-7-methoxy-6-(3-morpholin-4-ylpropoxy)quinazolin-4-amine (Iressa®), erlotinib N-(3-ethynylphenyl)-6,7-bis(2-methoxyethoxy)quinazolin-4-amine (Tarceva®) and EKB-569.

8. The method of claim 6, wherein the antibody is selected from the group consisting of certuximab (Erbitux®), panitumumab (Vectibix™) and EMD7200.

9. The method of claim 1, wherein the patient is further subject to androgen ablation treatment.

10. The method of claim 9, wherein androgen ablation treatment comprises administrating an antiandrogen.

11. The method of claim 1, wherein the antiandrogen is selected from the group consisting of flutamide (Eulexin), bicalutamide (Casodex) and nilutamide (Nilandron), leuprolin (Enanton Depot®), buserelin (Suprefact depot) and triptorelin (Pamorelin®).

12-22. (canceled)

23. A pharmaceutical composition comprising an EGFR inhibitor or EGFR signaling inhibitor or inhibitor of kinases downstream of EGFR kinases for treatment of metastatic prostate cancer.

24. The pharmaceutical composition according to claim 23, wherein the treatment is initiated while the primary tumor is in the androgen-sensitive stage of the disease.

25. The pharmaceutical composition according to claim 23, wherein the metastatic prostate cancer is in the androgen-sensitive stage of the disease.

26. The pharmaceutical composition according to claim 23, wherein the metastatic prostate cancer is skeletal metastatic prostate cancer.

27. The pharmaceutical composition according to claim 23, wherein the treatment is an adjuvant treatment following removal (surgery, radiotherapy, other therapy modajities) of the primary tumor.

28. The pharmaceutical composition according to claim 23, wherein the EGFR inhibitor or EGFR signaling inhibitor or inhibitor of kinases downstream of EGFR kinases is selected from the group consisting of small molecules, an antibody directed toward EGFR inhibitor or EGFR signaling inhibitor or inhibitor of kinases downstream of EGFR kinases and an aptamer directed toward EGFR inhibitor or EGFR signaling inhibitor or inhibitor of kinases downstream of EGFR kinases.

29. The pharmaceutical composition according to claim 28, wherein the small molecule is selected from the group consisting of gefitinib N-(3-chloro-4-fluoro-phenyl)-7-methoxy-6-(3-morpholin-4-ylpropoxy)quinazolin-4-amine (Iressa®), erlotinib N-(3-ethynylphenyl)-6,7-bis(2-methoxyethoxy)quinazolin-4-amine (Tarceva®) and EKB-569.

30. The pharmaceutical composition according to claim 28, wherein the antibody is selected from the group consisting of certuximab (Erbitux®), panitumumab (Vectibix™) and EMD7200.

31. The pharmaceutical composition according to claim 23, wherein the patient is further subject to androgen ablation treatment.

32. The pharmaceutical composition according to claim 31, wherein androgen ablation treatment comprises administrating an antiandrogen.

33. The pharmaceutical composition according to claim 32, wherein the antiandrogen is selected from the group consisting of flutamide (Eulexin), bicalutamide (Casodex) and nilutamide (Nilandron), leuprolin (Enanton Depot®), buserelin (Suprefact depot) and triptorelin (Pamorelin®).

34. A method of detecting metastatic prostate cancer, predicting metastatic prostate cancer or monitoring treatment of metastatic prostate cancer in an individual comprising
a) Providing a sample from the individual;
b) determining, in the sample, a level of EGFR protein, a level of EGFR phosphorylation, a level of EGFR kinase activity, a level of an EGFR pathway related protein, a level of an EGFR pathway related protein kinase activity, and/or a phosphorylation level of an EGFR pathway related protein; and
c) comparing the level(s) determined in step b with (a) control value(s) of control profiles.

35. A method of predicting an individual's response on EGFR inhibitor(s) or pharmaceutical composition(s), i.e. ex-vivo drug testing and response prediction based on validated biomarker(s) or biomarker profiles, comprising
a) Providing a sample from the individual;
b) determining, in the sample, a level of EGFR protein, a level of EGFR phosphorylation, a level of EGFR kinase activity, a level of an EGFR pathway related protein, a level of an EGFR pathway related protein kinase activity, a profile of EGFR pathway related protein kinase activities and/or a phosphorylation level of an EGFR pathway related protein; and
c) comparing the level(s) or profile determined in step b with (a) control value(s) or control profile from which the clinical outcome of the treatment can is a priori known.

36. The method of claim 34, wherein the control value(s) provided by determining the level of EGFR inhibitor or EGFR signaling inhibitor or inhibitor of kinases downstream of EGFR kinases in one or more healthy individuals.

37. The method of claim 34, wherein the read-out of control values comprises all or a subset of the set of peptide phosphorylations.

38. A method is provided for screening an individual for metastatic prostate cancer, the method comprising:
a) determining, in the sample of said individual, a level of EGFR protein, a level of
EGFR phosphorylation, a level of EGFR kinase activity, a level of an EGFR pathway related protein, a level of an EGFR pathway related protein kinase activity, and/or a phosphorylation level of an EGFR pathway related protein; and
b) constructing a percentile plot of EGFR concentrations, levels of EGFR phosphorylation, levels of EGFR kinase activity, concentrations of an EGFR pathway related protein, levels of phosphorylation of an EGFR pathway related protein and/or levels of an EGFR pathway related protein kinase activity, in a non-metastatic prostate cancer population;
c) constructing a ROC (receiver operating characteristics) curve based on EGFR concentrations, levels of EGFR phosphorylation, levels of EGFR kinase activity, concentrations of an EGFR pathway related, levels of phosphorylation of an EGFR pathway related protein and/or levels of an EGFR pathway related protein kinase activity determined in a non-metastatic prostate cancer population and on EGFR concentrations, levels of EGFR phosphorylation, levels of EGFR kinase activity, concentrations of an EGFR pathway related protein, levels of phosphorylation of an EGFR pathway related protein and/or levels of an EGFR pathway related protein kinase activity determined in a metastatic prostate cancer population;
d) selecting a desired sensitivity;
e) determining from the ROC curve the specificity corresponding to the desired sensitivity;
f) determining from the percentile plot the EGFR concentration value, the EGFR phosphorylation level value, the EGFR kinase activity value, the concentration value of an EGFR pathway related protein, the phosphorylation level value of an EGFR pathway related protein and/or the EGFR pathway related protein kinase activity levels value corresponding to the determined specificity; and
g) indicating the individual as likely to have metastatic prostate cancer if the concentration of EGFR, the levels of EGFR phosphorylation, the levels of EGFR kinase activity, the concentrations of an EGFR pathway related protein, the levels of phosphorylation of an EGFR pathway related protein and/or the levels of an EGFR pathway related protein kinase activity in the sample of the individual is equal to or higher than said EGFR concentration value, the EGFR phosphorylation level value, the EGFR kinase activity value, the concentration value of an EGFR pathway related protein, the phosphorylation level value of an EGFR pathway related protein and/or the EGFR pathway related protein kinase activity levels value corresponding to the determined specificity, and indicating the individual as unlikely to have metastatic prostate cancer if the concentration of EGFR, the levels of EGFR phosphorylation, the levels of EGFR kinase activity, the concentrations of an EGFR pathway related protein, the levels of phosphorylation of an EGFR pathway related protein and/or the levels of an EGFR pathway related protein kinase activity in the sample of the individual is lower than the EGFR concentration value, the EGFR phosphorylation level value, the EGFR kinase activity value, the concentration value of an EGFR pathway related protein, the phosphorylation level value of an EGFR pathway related protein and/or the EGFR pathway related protein kinase activity levels value corresponding to the determined specificity.

39. A method for screening an individual for metastatic prostate cancer, the method comprising:
a) determining, in the sample of said individual, a level of EGFR protein, a level of EGFR phosphorylation, a level of EGFR kinase activity, a level of an EGFR pathway related protein, a level of an EGFR pathway related protein kinase activity, and/or a phosphorylation level of an EGFR pathway related protein; and
b) constructing a percentile plot of EGFR concentrations, levels of EGFR phosphorylation, levels of EGFR kinase activity, concentrations of an EGFR pathway related protein, levels of phosphorylation of an EGFR pathway related protein and/or levels of an EGFR pathway related protein kinase activity determined in a non-metastatic prostate cancer population and on EGFR concentrations, levels of EGFR phosphorylation, levels of EGFR kinase activity, concentrations of an EGFR pathway related protein, levels of phosphorylation of an EGFR pathway related protein and/or levels of an EGFR pathway related protein kinase activity determined in a metastatic prostate cancer population;
c) selecting a desired specificity;
d) determining from the percentile plot the EGFR concentration value, the EGFR phosphorylation level value, the EGFR kinase activity value, the concentration value of an EGFR pathway related protein, the phosphorylation level value of an EGFR pathway related protein and/or the EGFR pathway related protein kinase activity levels value corresponding to the desired specificity; and
e) indicating the individual as likely to have metastatic prostate cancer if the concentration of EGFR, the levels of EGFR phosphorylation, the levels of EGFR kinase activity, the concentrations of an EGFR pathway related protein, the levels of phosphorylation of an EGFR pathway related protein and/or the levels of an EGFR pathway related protein kinase activity in the sample of the individual is equal to or higher than said EGFR concentration value the EGFR phosphorylation level value, the EGFR kinase activity value, the concentration value of an EGFR pathway related protein, the phosphorylation level value of an EGFR pathway related protein and/or the EGFR pathway related protein kinase activity levels value corresponding to the determined specificity, and indicating the individual as unlikely to have metastatic prostate cancer if the concentration of EGFR, the levels of EGFR phosphorylation, the levels of EGFR kinase activity, the concentrations of an EGFR pathway related protein, the levels of phosphorylation of an EGFR pathway related protein and/or the levels of an EGFR pathway related protein kinase activity in the sample of the individual is lower than the EGFR concentration value, the EGFR phosphorylation level value, the EGFR kinase activity value, the concentration value of an EGFR pathway related protein, the phosphorylation level value of an EGFR pathway related protein and/or the EGFR pathway related protein kinase activity levels value corresponding to the determined specificity.

40. A method for screening an individual for metastatic prostate cancer, the method comprising determining in a sample from said individual a level of EGFR protein, a level of EGFR phosphorylation, a level of EGFR kinase activity, a level of an EGFR pathway related protein, a level of an EGFR pathway related protein kinase activity, and/or a phosphorylation level of an EGFR pathway related protein, and indicating the individual as likely to have metastatic prostate cancer if said level is equal to or higher than the respective level measured in a non-metastatic prostate cancer population, and indicating the individual as unlikely to have metastatic prostate cancer if said level is lower than the respective level in a non-metastatic prostate cancer population.

41. A method according to claim 34, wherein the individual is a member of a population not already identified as having an increased risk of developing (metastatic) prostate cancer.

42. A method according to claim 34, wherein the individual is a member of a population not already identified as having an increased risk of developing (metastatic) prostate cancer.

43. A method according to claim 34, wherein the individual is a member of a population already identified as having an increased risk of developing (metastatic) prostate cancer.

44. A method according to claim 34, wherein the individual has a genetic disposition for (metastatic) prostate cancer, has been exposed to carcinogenic substances or has a (metastatic) prostate cancer-predisposing non-malignant disease.

45. A method according to claim 34, wherein the individual is selected from the group consisting of an individual who had any types of precursors to (metastatic) prostate cancer, and an individual with one or more family members with (metastatic) prostate cancer.

46. A method according to claim 34, wherein the metastatic prostate cancer is selected from the group consisting of localized disease, either androgen sensitive or androgen resistant.

47. The method according to claim 34, wherein the sample is provided from bone marrow of the patient.

48. The method according to claim 34, wherein the sample is provided from the blood.

49. The method according to claim 34, wherein the sample is provided from the tissue biopsies.

50. The Method according to claim 34, wherein said testing of EGFR protein in the sample and/or determining EGFR kinase activity in the sample and/or determining EGFR related kinase activity profiles by testing multiple markers assaying is by microarray analysis.

51. The method according to claim 34, wherein the determination of the concentration of EGFR in a sample of the individual is performed by means of an immunoassay or an activity assay.

52. The method according to claim 51, wherein the immunoassay is an ELISA.

53. The method according to claim 51, wherein the activity assay is zymography.

54. The method according to claim 51, wherein the immunoassay is western-blot or cytochemistry.

55. The method according to claim 50, wherein said microarray analysis is a three-dimensional flow-through solid support comprising through-going channels.

56. The method according to claim 55, wherein said solid support is a metal oxide support.

57. The method according to claim 34, wherein the method comprises determining:
i) the level of EGFR protein, the level of EGFR phosphorylation, and/or the level of EGFR kinase activity; and
ii) the level of at least one EGFR pathway related protein, the level of at least one EGFR pathway related protein kinase activity, and/or the phosphorylation level of at least one EGFR pathway related protein.
